SEQUENCE LISTING (50 sequences)

SEQ ID NO: 1 - protein, 108 aa, Mus musculus
QIVLTQSPAIMSASLGERVTMTCTASSSVSSSYLHWYQQKPGSSPKLWIYSTSNLASGVPARFSGSGSGTSYSLTISSMEAEDAATYYCHQYHRSPPTFGSGTKLEIK

SEQ ID NO: 2 - protein, 117 aa, Mus musculus
EVQLQQSGAELVRPGASVKLSCTASGFNIKDYYIHWVNQRPEQGLEWIGRIDPEDGDIAYAPKFQDKATLTVDTSSNTAYLHIIGLTSEDTAVYYCTTGNYYAMDFWGQGTSVTVSS

SEQ ID NO: 3 - protein, 12 aa, Mus musculus
TASSSVSSSYLH

SEQ ID NO: 4 - protein, 6 aa, Mus musculus
STSNLA

SEQ ID NO: 5 - protein, 9 aa, Mus musculus
HQYHRSPPT

SEQ ID NO: 6 - protein, 5 aa, Mus musculus
DYYIH

SEQ ID NO: 7 - protein, 17 aa, Mus musculus
RIDPEDGDIAYAPKFQD

SEQ ID NO: 8 - protein, 8 aa, Mus musculus
GNYYAMDF

SEQ ID NO: 9 - protein, 108 aa, synthetic construct
  REGION 1..108: note = humanized mouse IgG
EIVLTQSPATLSLSPGERVTLSCTASSSVSSSYLHWYQQKPGQSPRLWIYSTSNLASGVPARFSGSGPGTSFTLTISSLEPEDFAVYYCHQYHRSPPTFGQGTKLEIK

SEQ ID NO: 10 - protein, 108 aa, synthetic construct
  REGION 1..108: note = humanized mouse IgG
EIVLTQSPATLSLSPGERVTLSCTASSSVSSSYLHWYQQKPGSSPKLWIYSTSNLASGVPARFSGSGPGTSFTLTISSLEPEDFAVYYCHQYHRSPPTFGQGTKLEIK

SEQ ID NO: 11 - protein, 108 aa, synthetic construct
  REGION 1..108: note = humanized mouse IgG
EIVLTQSPATLSLSPGERVTLSCTASSSVSSSYLHWYQQKPGSSPKLWIYSTSNLASGVPARFSGSGPGTSYTLTISSMEPEDFAVYYCHQYHRSPPTFGQGTKLEIK

SEQ ID NO: 12 - protein, 108 aa, synthetic construct
  REGION 1..108: note = humanized mouse IgG
QIVLTQSPATLSASPGERVTLSCTASSSVSSSYLHWYQQKPGSSPKLWIYSTSNLASGVPARFSGSGPGTSYTLTISSMEPEDAATYYCHQYHRSPPTFGQGTKLEIK

SEQ ID NO: 13 - protein, 117 aa, synthetic construct
  REGION 1..117: note = humanized mouse IgG
EVQLVQSGAEVKKPGATVKISCKASGFNIKDYYIHWVNQAPGKGLEWIGRIDPEDGDIAYAPKFQDRVTLTVDTSTDTAYLELSSLRSEDTAVYYCTTGNYYAMDFWGQGTTVTVSS

SEQ ID NO: 14 - protein, 117 aa, synthetic construct
  REGION 1..117: note = humanized mouse IgG
EVQLVQSGAEVKKPGATVKLSCKASGFNIKDYYIHWVNQAPGKGLEWIGRIDPEDGDIAYAPKFQDRATLTVDTSTNTAYLELSSLTSEDTAVYYCTTGNYYAMDFWGQGTTVTVSS

SEQ ID NO: 15 - protein, 117 aa, synthetic construct
  REGION 1..117: note = humanized mouse IgG
EVQLQQSGAEVKKPGATVKLSCKASGFNIKDYYIHWVNQAPGKGLEWIGRIDPEDGDIAYAPKFQDRATLTVDTSTNTAYLELSSLRSEDTAVYYCTTGNYYAMDFWGQGTTVTVSS

SEQ ID NO: 16 - protein, 117 aa, synthetic construct
  REGION 1..117: note = humanized mouse IgG
EVQLQQSGAEVKKPGATVKLSCKASGFNIKDYYIHWVNQRPGQGLEWIGRIDPEDGDIAYAPKFQDRATLTVDTSTNTAYLELSSLRSEDTAVYYCTTGNYYAMDFWGQGTTVTVSS

SEQ ID NO: 17 - protein, 117 aa, synthetic construct
  REGION 1..117: note = humanized mouse IgG
EVQLQQSGAEVKKPGATVKLSCKASGFNIKDYYIHWVNQRPGQGLEWIGRIDPEDGDIAYAPKFQDRATLTVDTSTNTAYLELSSLTSEDTAVYYCTTGNYYAMDFWGQGTTVTVSS

SEQ ID NO: 18 - protein, 106 aa, synthetic construct
  REGION 1..106: note = humanized mouse IgG
EIVLTQSPDFQSVTPKEKVTITCRASSSVSYIHWYQQKPDSSPKAWISATSNLASGVPSRFSGSGSGTDFTLTINSLEAEDAATYYCQQWSSNPFTFGQGTKLEIK

SEQ ID NO: 19 - protein, 106 aa, synthetic construct
  REGION 1..106: note = humanized mouse IgG
EIVLTQSPDFQSATPKEKVTITCRASSSVSYIHWYQQKPDSSPKAWISATSNLASGVPSRFSGSGSGTSFTLTINSLEAEDAATYYCQQWSSNPFTFGQGTKLEIK

SEQ ID NO: 20 - protein, 106 aa, synthetic construct
  REGION 1..106: note = humanized mouse IgG
EIVLTQSPDFQSATPKEKVTMTCRASSSVSYIHWYQQKPDSSPKAWISATSNLASGVPSRFSGSGSGTSYTLTINSVEAEDAATYYCQQWSSNPFTFGQGTKLEIK

SEQ ID NO: 21 - protein, 106 aa, synthetic construct
  REGION 1..106: note = humanized mouse IgG
EIVLTQSPDFQSATPKEKVTITCRASSSVSYIHWYQQKPGSSPKAWISATSNLASGVPSRFSGSGSGTSYTLTINRVEAEDAATYYCQQWSSNPFTFGQGTKLEIK

SEQ ID NO: 22 - protein, 121 aa, synthetic construct
  REGION 1..121: note = humanized mouse IgG
QVQLVQSGAEVKKPGASVKVSCKASGYTFTFYTMHWVRQAPGQGLEWIGYINPSSGYTNYNQKFTDRVTLTADTSTSTAYMELSSLRSEDTAVYYCARSDGSSSKWYFDVWGQGTTVTVSS

SEQ ID NO: 23 - protein, 121 aa, synthetic construct
  REGION 1..121: note = humanized mouse IgG
QVQLVQSGAEVKKPGASVKVSCKASGYTFTFYTMHWLRQAPGQGLEWIGYINPSSGYTNYNQKFTDRATLTADTSTSTAYMELSSLRSEDTAVYYCARSDGSSSKWYFDVWGQGTTVTVSS

SEQ ID NO: 24 - protein, 121 aa, synthetic construct
  REGION 1..121: note = humanized mouse IgG
QVQLVQSGAEVKKPGASVKMSCKASGYTFTFYTMHWLKQAPGQGLEWIGYINPSSGYTNYNQKFTDRATLTADTSTSTAYMELSSLRSEDTAVYYCARSDGSSSKWYFDVWGQGTTVTVSS

SEQ ID NO: 25 - protein, 121 aa, synthetic construct
  REGION 1..121: note = humanized mouse IgG
QVQLVQSGAEVKKPGASVKMSCKASGYTFTFYTMHWLKQAPGQGLEWIGYINPSSGYTNYNQKFTDRATLTADKSTSTAYMELSSLRSEDTAVYYCARSDGSSSKWYFDVWGQGTTVTVSS

SEQ ID NO: 26 - protein, 10 aa, Mus musculus
RASSSVSYIH

SEQ ID NO: 27 - protein, 7 aa, Mus musculus
ATSNLAS

SEQ ID NO: 28 - protein, 9 aa, Mus musculus
QQWSSNPFT

SEQ ID NO: 29 - protein, 5 aa, Mus musculus
FYTMH

SEQ ID NO: 30 - protein, 17 aa, Mus musculus
YINPSSGYTNYNQKFTD

SEQ ID NO: 31 - protein, 12 aa, Mus musculus
SDGSSSKWYFDV

SEQ ID NO: 32 - protein, 443 aa, synthetic construct
  REGION 1..443: note = humanized mouse Fv, human Fc
EVQLVQSGAEVKKPGATVKISCKASGFNIKDYYIHWVNQAPGKGLEWIGRIDPEDGDIAYAPKFQDRVTLTVDTSTDTAYLELSSLRSEDTAVYYCTTGNYYAMDFWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 33 - protein, 215 aa, synthetic construct
  REGION 1..215: note = humanized mouse Fv, human constant (Ckappa)
QIVLTQSPATLSASPGERVTLSCTASSSVSSSYLHWYQQKPGSSPKLWIYSTSNLASGVPARFSGSGPGTSYTLTISSMEPEDAATYYCHQYHRSPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 443 aa, synthetic construct
  REGION 1..443: note = humanized mouse Fv, human Fc
EVQLQQSGAEVKKPGATVKLSCKASGFNIKDYYIHWVNQRPGQGLEWIGRIDPEDGDIAYAPKFQDRATLTVDTSTNTAYLELSSLRSEDTAVYYCTTGNYYAMDFWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 35 - protein, 106 aa, Mus musculus
QIVLSQSPTILSASPGEKVTMTCRASSSVSYIHWYQQKPGSSPKAWISATSNLASGVPARFSGSGSGTSYSLTISRVEAEDAATYYCQQWSSNPFTFGSGTKLEIK

SEQ ID NO: 36 - protein, 121 aa, Mus musculus
QVQLQQSGAELARPGASVKMSCKASGYTFTFYTMHWLKQRPGQGLEWIGYINPSSGYTNYNQKFTDKATLTADKSSSTAYMQLSSLTSEDSAVYYCARSDGSSSKWYFDVWGTGTTVTVSS

SEQ ID NO: 37 - protein, 215 aa, synthetic construct
  REGION 1..215: note = humanized mouse Fv, human constant (Ckappa)
EIVLTQSPATLSLSPGERVTLSCTASSSVSSSYLHWYQQKPGQSPRLWIYSTSNLASGVPARFSGSGPGTSFTLTISSLEPEDFAVYYCHQYHRSPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 215 aa, synthetic construct
  REGION 1..215: note = humanized mouse Fv, human constant (Ckappa)
EIVLTQSPATLSLSPGERVTLSCTASSSVSSSYLHWYQQKPGSSPKLWIYSTSNLASGVPARFSGSGPGTSFTLTISSLEPEDFAVYYCHQYHRSPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 215 aa, synthetic construct
  REGION 1..215: note = humanized mouse Fv, human constant (Ckappa)
EIVLTQSPATLSLSPGERVTLSCTASSSVSSSYLHWYQQKPGSSPKLWIYSTSNLASGVPARFSGSGPGTSYTLTISSMEPEDFAVYYCHQYHRSPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 443 aa, synthetic construct
  REGION 1..443: note = humanized mouse Fv, human Fc
EVQLVQSGAEVKKPGATVKLSCKASGFNIKDYYIHWVNQAPGKGLEWIGRIDPEDGDIAYAPKFQDRATLTVDTSTNTAYLELSSLTSEDTAVYYCTTGNYYAMDFWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 41 - protein, 443 aa, synthetic construct
  REGION 1..443: note = humanized mouse Fv, human Fc
EVQLQQSGAEVKKPGATVKLSCKASGFNIKDYYIHWVNQAPGKGLEWIGRIDPEDGDIAYAPKFQDRATLTVDTSTNTAYLELSSLRSEDTAVYYCTTGNYYAMDFWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 42 - protein, 443 aa, synthetic construct
  REGION 1..443: note = 42 humanized mouse Fv, human Fc
EVQLQQSGAEVKKPGATVKLSCKASGFNIKDYYIHWVNQRPGQGLEWIGRIDPEDGDIAYAPKFQDRATLTVDTSTNTAYLELSSLTSEDTAVYYCTTGNYYAMDFWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 43 - protein, 213 aa, synthetic construct
  REGION 1..213: note = humanized mouse Fv, human constant (Ckappa)
EIVLTQSPDFQSVTPKEKVTITCRASSSVSYIHWYQQKPDSSPKAWISATSNLASGVPSRFSGSGSGTDFTLTINSLEAEDAATYYCQQWSSNPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 213 aa, synthetic construct
  REGION 1..213: note = 44 humanized mouse Fv, human constant (Ckappa)
EIVLTQSPDFQSATPKEKVTITCRASSSVSYIHWYQQKPDSSPKAWISATSNLASGVPSRFSGSGSGTSFTLTINSLEAEDAATYYCQQWSSNPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 213 aa, synthetic construct
  REGION 1..213: note = humanized mouse Fv, human constant (Ckappa)
EIVLTQSPDFQSATPKEKVTMTCRASSSVSYIHWYQQKPDSSPKAWISATSNLASGVPSRFSGSGSGTSYTLTINSVEAEDAATYYCQQWSSNPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 213 aa, synthetic construct
  REGION 1..213: note = 46 humanized mouse Fv, human constant (Ckappa)
EIVLTQSPDFQSATPKEKVTITCRASSSVSYIHWYQQKPGSSPKAWISATSNLASGVPSRFSGSGSGTSYTLTINRVEAEDAATYYCQQWSSNPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 447 aa, synthetic construct
  REGION 1..447: note = humanized mouse Fv, human Fc
QVQLVQSGAEVKKPGASVKVSCKASGYTFTFYTMHWVRQAPGQGLEWIGYINPSSGYTNYNQKFTDRVTLTADTSTSTAYMELSSLRSEDTAVYYCARSDGSSSKWYFDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 48 - protein, 447 aa, synthetic construct
  REGION 1..447: note = humanized mouse Fv, human Fc
QVQLVQSGAEVKKPGASVKVSCKASGYTFTFYTMHWLRQAPGQGLEWIGYINPSSGYTNYNQKFTDRATLTADTSTSTAYMELSSLRSEDTAVYYCARSDGSSSKWYFDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 49 - protein, 447 aa, synthetic construct
  REGION 1..447: note = humanized mouse Fv, human Fc
QVQLVQSGAEVKKPGASVKMSCKASGYTFTFYTMHWLKQAPGQGLEWIGYINPSSGYTNYNQKFTDRATLTADTSTSTAYMELSSLRSEDTAVYYCARSDGSSSKWYFDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 50 - protein, 447 aa, synthetic construct
  REGION 1..447: note = humanized mouse Fv, human Fc
QVQLVQSGAEVKKPGASVKMSCKASGYTFTFYTMHWLKQAPGQGLEWIGYINPSSGYTNYNQKFTDRATLTADKSTSTAYMELSSLRSEDTAVYYCARSDGSSSKWYFDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

(19) United States
(12) Patent Application Publication
(10) Pub. No.: US 2025/0145727 A1
(43) Pub. Date: May 8, 2025

(54) CD137 AGONIST ANTIBODIES AND USES THEREOF

(71) Applicant: QLSF BIOTHERAPEUTICS INC., South San Francisco, CA (US)

(72) Inventors: Aaron Kurtzman, South San Francisco, CA (US); Hieu Tran, South San Francisco, CA (US); Shihao Chen, South San Francisco, CA (US)

(21) Appl. No.: 19/019,453

(22) Filed: Jan. 13, 2025

Related U.S. Application Data
(63) Continuation of application No. 17/275,153, filed on Mar. 10, 2021, now Pat. No. 12,227,582.
(60) Provisional application No. 62/787,509, filed on Jan. 2, 2019.

(51) Int. Cl. C07K 16/28; A61K 47/68; A61P 35/00

(52) U.S. Cl. CPC C07K 16/2878; A61K 47/6849; A61P 35/00; C07K 2317/24; C07K 2317/75; C07K 2317/92

(57) ABSTRACT

The present disclosure provides isolated binding molecules that bind to and activates CD137, vectors comprising a nucleic acid molecules encoding an amino acid sequence of the binding molecules, host cells containing the vectors, methods of making the binding molecules, pharmaceutical compositions containing the binding molecules, and methods of using such antibodies, antibody fragments and derivatives and polypeptides, including methods of treating a disease requiring stimulation of immune responses including cancer.

### CROSS REFERENCE TO RELATED APPLICATION

This application is a continuation of U.S. Nonprovisional patent application Ser. No. 17/275,153 filed on Mar. 10, 2021, which a 371 International of PCT/US20/12080 filed Jan. 2, 2020, which claims the benefit of U.S. Provisional Application No. 62/787,509, filed Jan. 2, 2019. The disclosure of each of these prior-filed applications is incorporated herein in its entirety.

### INCORPORATION OF SEQUENCE LISTING

This application includes a Sequence Listing which is being submitted in XML format via Patent Center, named “QLSF001US_CON1.XML,” which is 64 KB in size and created on Jan. 1, 2025. The contents of the Sequence Listing are incorporated herein by reference in their entirety.

### BACKGROUND OF THE INVENTION

CD137 is a member of the Tumor Necrosis Factor (TNF) receptor family. Its alternative names are Tumor Necrosis Factor Receptor Superfamily member 9 (TNFRSF9), 4-1BB and Receptor Induced by Lymphocyte Activation (ILA). CD137 can be expressed by activated T cells, but to a larger extent on CD8 than on CD4 T cells. In addition, the CD137 expression is found on dendritic cells, follicular dendritic cells, natural killer cells, granulocytes and cells of blood vessel walls at sites of inflammation. One characteristic activity of CD137 is its costimulatory activity for activated T cells. Crosslinking of CD137 enhances T cell proliferation, IL-2 secretion survival and cytolytic activity. Further, it can enhance immune activity to eliminate tumors in mice.

CD137 can be induced on TCR activation as a T-cell costimulatory receptor (Nam et al., Curr. Cancer Drug Targets,5:357-363 (2005); Watts et al., Annu. Rev. Immunol., 23:23-68 (2005)). In addition to its expression on activated CD4+ and CD8+ T cells, CD137 is also expressed on CD4+CD25+ regulatory T cells. Its natural ligand, CD137L, has been described on antigen-presenting cells including B cells, monocyte/macrophages, and dendritic cells (Watts et al., Annu. Rev. Immunol., 23:23-68 (2005)).

Signaling through CD137 by either CD137L or agonistic monoclonal antibodies (mAbs) binding CD137 leads to increased TCR-induced T cell proliferation, cytokine production and functional maturation, and prolonged CD8+T cell survival. These effects result from: (1) the activation of the NF-KB, c-Jun NH2-terminal kinase/stress-activated protein kinase (JNK/SAPK), and p38 mitogen-activated protein kinase (MAPK) signaling pathways, and (2) the control of anti-apoptotic and cell cycle-related gene expression. Experiments performed in both CD137 and CD137L-deficient mice have additionally demonstrated the importance of CD137 costimulation in the generation of a fully competent T cell response.

IL-2 and IL-15 activated NK cells express CD137, and ligation of CD137 by agonistic mAbs stimulates NK cell proliferation and IFN-y secretion, but not their cytolytic activity. Furthermore, CD137-stimulated NK cells promote the expansion of activated T cells in vitro. In accordance with their costimulatory function, agonist mAbs against CD137 have been shown to promote rejection of cardiac and skin allografts, eradicate established tumors, broaden primary antiviral CD8+ T cell responses, and increase T cell cytolytic potential. These studies support the view that CD137 signaling promotes T cell function which may enhance immunity against tumors and infection.

Anti-CD137 antibodies have been disclosed in U.S. 2005/0095244, issued U.S. Pat. No. 7,288,638 (such as 20H4.9-IgG4 [10C7 or BMS-663513] or 20H4.9-IgG1 [BMS-663031]); U.S. Pat. No. 6,887,673 [cxE9 or BMS-554271]; U.S. Pat. Nos. 7,214,493; 6,303,121; 6,569,997; 6,905,685; 6,355,476; 6,362,325 [IDS or BMS-469492; 3H3 or BMS-469497; or 3E1]; U.S. Pat. No. 6,974,863 (such as 53A2); or U.S. Pat. No. 6,210,669 (such as IDS, 3B8, or 3E1), or U.S. Pat. No. 8,337,850. Additional CD137 agonistic antibodies are described in U.S. 2016/0244528, U.S. Pat. Nos. 5,928,893; 6,303,121, 6,569,997, and 8,137,667.

### SUMMARY OF THE INVENTION

The present disclosure provides isolated monoclonal anti-CD137 agonist antibodies, and antigen-binding portions thereof that specifically bind to human CD137.

In an aspect of the invention, an isolated monoclonal anti-CD137 agonist antibody, or antigen-binding portion thereof comprises a heavy chain variable region CDR3 comprising SEQ ID NO:8. In some embodiments, the monoclonal anti-CD137 agonist antibody, or antigen-binding portion thereof further comprises a heavy chain variable region CDR1 comprising SEQ ID NO:6 and a heavy chain variable region CDR2 comprising SEQ ID NO:7. In preferred embodiments, the monoclonal anti-CD137 agonist antibody, or antigen-binding portion thereof further comprises: (a) a light chain variable region CDR1 comprising SEQ ID NO:3; (b) a light chain variable region CDR2 comprising SEQ ID NO:4; and (c) a light chain variable region CDR3 comprising SEQ ID NO: 5. In one embodiment, the antibody or portion comprises a light chain variable region amino acid sequence having at least 95% identity to SEQ ID NO:1 and a heavy chain variable region amino acid sequence having at least 95% identity to SEQ ID NO:2. In another embodiment, the antibody or portion comprises a heavy chain variable region comprising an amino acid sequence that has at least 95%, at least 96%, at least 97%, at least 98%, or at least 99% identical to a sequence as set forth in SEQ ID NO:2.

In an aspect of the invention, an isolated monoclonal anti-CD137 agonist antibody, or antigen-binding portion thereof comprises a heavy chain variable region CDR3 comprising SEQ ID NO:31. In some embodiments, the monoclonal anti-CD137 agonist antibody, or antigen-binding portion thereof further comprises a heavy chain variable region CDR1 comprising SEQ ID NO:29 and a heavy chain variable region CDR2 comprising SEQ ID NO:30. In preferred embodiments, the monoclonal anti-CD137 agonist antibody, or antigen-binding portion thereof further comprises: (a) a light chain variable region CDR1 comprising SEQ ID NO:26; (b) a light chain variable region CDR2 comprising SEQ ID NO:27; and (c) a light chain variable region CDR3 comprising SEQ ID NO: 28. In one embodiment, the antibody or portion comprises a light chain variable region amino acid sequence having at least 95% identity to SEQ ID NO:35 and a heavy chain variable region amino acid sequence having at least 95% identity to SEQ ID NO:36. In another embodiment, the antibody or portion comprises a heavy chain variable region comprising an amino acid sequence that is at least 95%, at least 96%, at least 97%, at least 98%, or at least 99% identical to a sequence as set forth in SEQ ID NO:36.

In another aspect of the invention, an isolated monoclonal anti-CD137 agonist antibody or an antigen-binding portion thereof comprises a heavy chain variable region comprising an amino sequence selected from the group consisting of SEQ ID NOs:22-25 and a light chain variable region comprising an amino sequence selected from the group consisting of SEQ ID NOs:18-21.

In another aspect of the invention, an isolated monoclonal anti-CD137 agonist antibody or an antigen-binding portion thereof comprises: a heavy chain variable region comprising an amino sequence selected from the group consisting of SEQ ID NOs: 13-17 and a light chain variable region comprising an amino sequence selected from the group consisting of SEQ ID NOs:9-12.

The antibodies of the disclosed invention can be further engineered into formats suitable for human therapeutics by modifications that minimize immunogenicity. Suitable antibodies include, but are not limited to chimeric antibodies and humanized antibodies. The affinity, stability and specificity of the disclosed antibodies can also be further optimized by techniques known to one of skill in the art. Other formats can involve oligomerization, drug conjugation and fusion of the disclosed antibodies with other functional proteins.

The antibodies of the disclosed invention can be, for example, full-length antibodies, for example of an IgG1, IgG2, IgG3, or IgG4 isotype. Alternatively, the disclosed antibodies can be antibody fragments, such as Fab, Fab′ and F(ab′)₂ fragments, diabody, triabody, tetrabody, single-chain variable region fragment (scFv), disulfide-stabilized variable region fragment (dsFv), and half antibodies. Alternatively, the disclosed antibodies can be bispecific antibodies.

In another aspect of the invention, an isolated monoclonal antibody or antigen binding portion thereof comprises a light chain comprises an amino acid sequence selected from the group consisting of SEQ ID NOs:33, 37-39 and a heavy chain comprising an amino acid sequence selected from the group consisting of SEQ ID NOs:32, 34, 40-42.

In another aspect of the invention, an isolated monoclonal antibody or antigen binding portion thereof comprises a light chain comprising an amino acid sequence selected from the group consisting of SEQ ID NOs:43-46 and a heavy chain comprising an amino acid sequence selected from the group consisting of SEQ ID NOs:47-50.

In some embodiments, the anti-CD137 agonist antibody, or antigen-binding portion thereof binds to and activates human CD137. Therefore the antibody, or antigen-binding portion can stimulate an anti-tumor immune response. In some embodiments, the anti-CD137 agonist antibody, or antigen-binding portion thereof binds to and activates non-human primate CD137. In some embodiments, the anti-CD137 agonist antibody, or antigen-binding portion thereof binds to and activates mammalian CD137.

In another aspect of the invention, a composition comprising the isolated anti-CD137 agonist monoclonal antibody, or antigen-binding portion thereof is also provided.

In another aspect of the invention, a pharmaceutical composition comprising the isolated anti-CD137 agonist monoclonal antibody, or antigen-binding portion thereof and a pharmaceutically acceptable carrier are also provided. Compositions comprising an immunoconjugate of the invention and a pharmaceutically acceptable carrier are also provided.

In another aspect of the invention, a vector comprising an isolated nucleic acid molecule encoding the antibody, or antigen-binding portion thereof, and a host cell comprising an expression vector comprising said nucleic acid molecule are also provided.

The present invention further provides a method of stimulating immune responses using the anti-CD137 agonist antibodies of the disclosed invention. For example, in one embodiment, the disclosed invention provides a method for treating a subject in need thereof, comprising the step of administering to the subject an effective amount of the antibody or antigen-binding portion of the disclosed invention.

In another aspect, the disclosed invention provides a method for treating cancer in a human comprising the step of administering to the human the anti-CD137 agonist antibody or antigen-binding portion of the disclosed invention in an amount effective to treat said cancer.

In another aspect, the disclosed invention provides a method for treating infectious diseases in a human comprising the step of administering to the human the anti-CD137 agonist antibody or antigen-binding portion of the disclosed invention in an amount effective to treat said infectious diseases.

Other features and advantages of the instant disclosure will be apparent from the following detailed description and examples, which should not be construed as limiting. The contents of all references,

GenBank entries, patents and published patent applications cited throughout this application are expressly incorporated herein by reference.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1A and 1B show anti-4-1BB (anti-CD137) agonist antibody of the present disclosure induces NF-KB reporter activation.

FIG. 2A and 2B show anti-4-1BB (anti-CD137) agonist antibody of the present disclosure stimulates primary CD3+ CD8+ T cell proliferation. FIG. 2C and 2D show anti-4-1BB (anti-CD137) agonist antibody stimulating primary CD3+ CD8− T cell proliferation.

FIG. 3A and 3B show anti-4-1BB (anti-CD137) agonist antibody of the present disclosure stimulates interferon-γ secretion.

FIG. 4A and 4B show anti-4-1BB (anti-CD137) agonist antibody of the present disclosure inhibits cancers in hu4-1BB knock-in mice.

### DETAILED DESCRIPTION OF THE INVENTION

The following embodiments and aspects thereof are described and illustrated in conjunction with systems, compositions and methods which are meant to be exemplary and illustrative, not limiting in scope.

### DEFINITIONS

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

As used herein, the term “about” refers to a measurable value such as an amount, a time duration, and the like, and encompasses variations of ±20%, ±10%, ±5%, ±1%, ±0.5% or ±0.1% from the specified value.

The term “epitope” as used herein can include any protein determinant capable of specific binding to an immunoglobulin or T-cell receptor. Epitopic determinants usually consist of chemically active surface groupings of molecules such as amino acids or sugar side chains and usually have specific three-dimensional structural characteristics, as well as specific charge characteristics. An antibody is said to specifically bind an antigen when the equilibrium dissociation constant is ≤1 μM, preferably ≤100 nM and most preferably ≤10 nM.

The term “K_{D}” can refer to the equilibrium dissociation constant of a particular antibody-antigen interaction.

The term “immune response” as used herein can refer to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from an organism of invading pathogens, cells or tissues infected with pathogens, cancerous cells, or, in cases of autoimmunity or pathological inflammation, normal organismal cells or tissues.

An “antigen-specific T cell response” as used herein can refer to responses by a T cell that result from stimulation of the T cell with the antigen for which the T cell is specific. Non-limiting examples of responses by a T cell upon antigen-specific stimulation include proliferation and cytokine production (e.g., IL-2 production).

As used herein, the term “antibody” refers to an intact immunoglobulin or to a monoclonal or polyclonal antigen-binding fragment with the Fc (crystallizable fragment) region or FcRn binding fragment of the Fc region, referred to herein as the “Fc fragment” or “Fc region”. Antigen-binding fragments may be produced by recombinant DNA techniques or by enzymatic or chemical cleavage of intact antibodies. Antigen-binding fragments include, inter alia, Fab, Fab′, F(ab′)2, Fv, dAb, and complementarity determining region (CDR) fragments, single-chain antibodies (scFv), single region antibodies, chimeric antibodies, diabodies and polypeptides that contain at least a portion of an immunoglobulin that is sufficient to confer specific antigen binding to the polypeptide. The Fc region includes portions of two heavy chains contributing to two or three classes of the antibody. The Fc region may be produced by recombinant DNA techniques or by enzymatic (e.g. papain cleavage) or via chemical cleavage of intact antibodies.

The term “antibody fragment,” as used herein, refers to a protein fragment that comprises only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 regions; (ii) the Fab′ fragment, which is a Fab fragment having one or more cysteine residues at the C-terminus of the CH1 region; (iii) the Fd fragment having VH and CH1 regions; (iv) the Fd′ fragment having VH and CH1 regions and one or more cysteine residues at the C-terminus of the CH1 region; (v) the Fv fragment having the VL and VH regions of a single arm of an antibody; (vi) the dAb fragment (Ward et al., Nature 341, 544-546 (1989)) which consists of a VH region; (vii) isolated CDR regions; (viii) F(ab′)2 fragments, a bivalent fragment including two Fab′ fragments linked by a disulfide bridge at the hinge region; (ix) single chain antibody molecules (e.g., single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable region (VH) connected to a light chain variable region (VL) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (VH-CH1-VH-CH1) which, together with complementary light chain polypeptides, form a pair of antigen binding regions (Zapata et al. Protein Eng. 8(10):1057-1062 (1995); and U.S. Pat. No. 5,641,870).

“Single-chain variable fragment”, “single-chain antibody variable fragments” or “scFv” antibodies as used herein refers to forms of antibodies comprising the variable regions of only the heavy (VH) and light (VL) chains, connected by a linker peptide. The scFvs are capable of being expressed as a single chain polypeptide. The scFvs retain the specificity of the intact antibody from which it is derived. The light and heavy chains may be in any order, for example, VH-linker-VL or VL-linker-VH, so long as the specificity of the scFv to the target antigen is retained.

An “isolated antibody”, as used herein, can refer to an antibody that is substantially free of other antibodies having different antigenic specificities (e.g., an isolated antibody that specifically binds a CD137 protein can be substantially free of antibodies that specifically bind antigens other than CD137 proteins). An isolated antibody that specifically binds a human CD137 protein can, however, have cross-reactivity to other antigens, such as CD137 proteins from other species. Moreover, an isolated antibody can be substantially free of other cellular material and/or chemicals.

Anti-CD137 agonist antibody-producing cells, e.g., hybridomas, can be selected, cloned and further screened for desirable characteristics, including robust growth, high antibody production and desirable antibody characteristics. Hybridomas can be expanded in vivo in syngeneic animals, in animals that lack an immune system, e.g., nude mice, or in cell culture in vitro. Methods of selecting, cloning and expanding hybridomas are well known to those of ordinary skill in the art.

The terms “monoclonal antibody” or “monoclonal antibody composition” as used herein can refer to a preparation of antibody molecules of single molecular composition. A monoclonal antibody composition displays a single binding specificity and affinity for a particular epitope.

The term “recombinant human antibody”, as used herein, can refer to all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “isotype” can refer to the antibody class (e.g., IgM or IgG1) that is encoded by the heavy chain constant region genes. An antibody can be an immunoglobulin G (IgG), an IgM, an IgE, an IgA or an IgD molecule, or is derived therefrom.

The phrases “an antibody recognizing an antigen” and “an antibody specific for an antigen” are used interchangeably herein with the term “an antibody which binds specifically to an antigen.”

As used herein, an antibody that “specifically binds human CD137” can refer to an antibody that binds to a human CD137 protein (and possibly a CD137 protein from one or more non-human species) but does not substantially bind to non-CD137 proteins. Preferably, the antibody binds to a human CD137 protein with “high affinity,” namely with a K_{D} of 1×10⁻⁷ M or less, more preferably 5×10⁻⁸ M or less, more preferably 3×10⁻⁸ M or less, more preferably 1×10⁻⁸ M or less, more preferably 5×10⁻⁹ M or less or even more preferably 1×10⁻⁹ M or less.

The term “does not substantially bind” to a protein or cells, as used herein, can mean that it cannot bind or does not bind with a high affinity to the protein or cells, i.e., binds to the protein or cells with an K_{D} of 2×10⁻⁶ M or more, more preferably 1×10⁻⁵ M or more, more preferably 1×10⁻⁴ M or more, more preferably 1×10⁻³ M or more, even more preferably 1×10⁻² M or more.

The term “high affinity” for an IgG antibody can refer to an antibody having a K_{D} of 1×10⁻⁶ M or less, preferably 1×10⁻⁷ M or less, more preferably 1×10⁻¹⁰ M or less, even more preferably 1×10⁻⁹ M or less, even more preferably 1×10⁻¹⁰ M or less for a target antigen. However, “high affinity” binding can vary for other antibody isotypes.

The term “pharmaceutical formulation” refers to a preparation which is in such form as to permit the biological activity of an active ingredient contained therein to be effective, and which contains no additional components which are unacceptably toxic to a subject to which the formulation would be administered.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmaco-dynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

An “agonist antibody” as used herein, is an antibody that induces or increases the biological activity of an antigen (for example, CD137) to which the antibody binds. An agonist may, for example, facilitate a receptor's phosphorylation due to binding of the receptor to a ligand or may activate or grow cells activated by the receptor. In one embodiment, the antibodies of the invention are agonist anti-CD137 antibodies.

A “CDR grafted antibody” is an antibody comprising one or more CDRs derived from an antibody of a particular species or isotype and the framework of another antibody of the same or different species or isotype.

A “humanized antibody” has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant regions of the heavy and/or light chains of the non-human species antibody are mutated to produce the humanized antibody. In another embodiment, the constant region(s) from a human antibody are fused to the variable region(s) of a non-human species. In another embodiment, a humanized antibody is a CDR grafted antibody comprising one or more CDRs derived from an antibody of a particular species or isotype and the framework of human antibodies. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen.

Examples of how to make humanized antibodies may be found in U.S. Pat. Nos. 6,054,297, 5,886,152 and 5,877,293.

The term “chimeric antibody” refers to an antibody that contains one or more regions from one antibody and one or more regions from one or more other antibodies. In one embodiment, one or more of the CDRs are derived from a human anti-CD137 antibody. In another embodiment, all of the CDRs are derived from a human anti-CD137 antibody. In another embodiment, the CDRs from more than one human anti-CD137 antibodies are mixed and matched in a chimeric antibody. For instance, a chimeric antibody may comprise a CDR1 from the light chain of a first human anti-CD137 antibody, a CDR2 and a CDR3 from the light chain of a second human anti-CD137 antibody, and the CDRs from the heavy chain from a third anti-CD137 antibody. Other combinations are possible.

The term “subject” can refer to any human or non-human animal. The subject can be male or female and can be any suitable age, including infant, juvenile, adolescent, adult, and geriatric subjects. The term “nonhuman animal” includes all vertebrates, e.g., mammals and non-mammals, such as nonhuman primates, sheep, dogs, cats, cows, horses, chickens, rabbits, mice, rats, amphibians, and reptiles, although mammals are preferred, such as non-human primates, sheep, dogs, cats, cows and horses.

The binding of an antibody of the disclosed invention to CD137 can be assessed using one or more techniques well established in the art. For example, in a preferred embodiment, an antibody can be tested by ELISA assays, for example using a recombinant CD137 protein. Still other suitable binding assays include but are not limited to a flow cytometry assay in which the antibody is reacted with a cell line that expresses human CD137, such as HEK293 cells that have been transfected to express CD137 (e.g., human CD137) on their cell surface. Additionally or alternatively, the binding of the antibody, including the binding kinetics (e.g., K_{D} value) can be tested in BIAcore binding assays, Octet Red96 (Pall) and the like.

Preferably, an antibody of the disclosed invention binds to a human CD137 protein with a K_{D} of 5×10⁻⁸ M or less, binds to a human CD137 protein with a K_{D} of 2×10⁻⁸ M or less, binds to a human CD137 protein with a K_{D} of 5×10⁻⁹ M or less, binds to a human CD137 protein with a K_{D} of 4×10⁻⁹ M or less, binds to a human CD137 protein with a K_{D} of 3×10⁻⁹ M or less, binds to a human CD137 protein with a K_{D} of 2×10⁻⁹ M or less, binds to a human CD137 protein with a K_{D} of 1×10⁻⁹ M or less.

The present disclosure relates to isolated monoclonal antibodies, or antigen binding portions thereof, which binds to and activates CD137, and uses thereof. In certain embodiments, the antibodies of the disclosed invention are derived from identified heavy and light chain germline sequences and/or comprise identified structural features such as CDR regions comprising identified amino acid sequences. This disclosure provides isolated antibodies, methods of making such antibodies and antigen-binding portions thereof of the disclosed invention. This disclosure also relates to methods of using the antibodies, such as using the anti-CD137 agonist antibodies of the disclosed invention to stimulate immune responses, alone or in combination with other immunostimulatory antibodies. Accordingly, also provided are methods of using the anti-CD137 agonist antibodies of the disclosed invention for example, including but not limited to, treating cancer in a human. Various aspects of the invention relate to antibodies and antibody fragments, pharmaceutical compositions, nucleic acids, recombinant expression vectors, and host cells for making such antibodies and fragments. Methods of using the antibodies of the invention to detect human CD137, to stimulate CD137 activity, either in vitro or in vivo, and to prevent or treat disorders such as cancer are also encompassed by the invention.

Complementarity determining regions (CDRs) are known as hypervariable regions both in the light chain and the heavy chain variable regions. The more highly conserved portions of variable regions are called the framework (FR). Complementarity determining regions (CDRs) and framework regions (FR) of a given antibody may be identified using the system described by Kabat et al. supra; Lefranc et al., supra and/or Honegger and Pluckthun, supra. Also familiar to those in the art is the numbering system described in Kabat et al. (1991, NIH Publication 91-3242, National Technical Information Service, Springfield, Va.). In this regard Kabat et al. defined a numbering system for variable region sequences that is applicable to any antibody. One of ordinary skill in the art can unambiguously assign this system of “Kabat numbering” to any variable region amino acid sequence, without reliance on any experimental data beyond the sequence itself.

In certain embodiments, the present invention provides anti-CD137 agonist antibodies or antigen-binding portions thereof. In one embodiment, the antibody or portion comprises (a) a light chain variable region CDR1 comprising SEQ ID NO:3; (b) a light chain variable region CDR2 comprising SEQ ID NO:4; (c) a light chain variable region CDR3 comprising SEQ ID NO:5; (d) a heavy chain variable region CDR1 comprising SEQ ID NO:6; (e) a heavy chain variable region CDR2 comprising SEQ ID NO:7; (f) a heavy chain variable region CDR3 comprising SEQ ID NO: 8. In another embodiment, the antibody or portion comprises (a) a light chain variable region CDR1 comprising SEQ ID NO:26; (b) a light chain variable region CDR2 comprising SEQ ID NO: 27; (c) a light chain variable region CDR3 comprising SEQ ID NO:28; (d) a heavy chain variable region CDR1 comprising SEQ ID NO:29; (e) a heavy chain variable region CDR2 comprising SEQ ID NO:30; (f) a heavy chain variable region CDR3 comprising SEQ ID NO:31.

In one embodiment, the present disclosure provides a monoclonal antibody or antigen-binding portion thereof that binds to a CD137 epitope, that comprises a light chain variable region amino acid sequence having at least 95% identity to SEQ ID NO:1 or 35 and a heavy chain variable region amino acid sequence having at least 95% identity to SEQ ID NO:2 or 36.

Given that each of these antibody Fab can bind to human CD137, the VH and VL sequences can be “mixed and matched” to create other anti-CD137 binding molecules of the invention. Preferably, when VH and VL chains are mixed and matched, a VH sequence from a particular VH/VL pairing is replaced with a structurally similar VH sequence. Likewise, preferably a VL sequence from a particular VH/VL pairing is replaced with a structurally similar VL sequence.

In some embodiments, the humanized antibody or antigen binding portion thereof comprises a heavy chain variable region comprising an amino acid sequence selected from the group consisting of SEQ ID NOs: 13-17 and a light chain variable region comprising an amino acid sequence selected from the group consisting of SEQ ID NOs: 9-12. Preferred heavy and light chain combinations include but not limited to:
(a) a heavy chain variable region comprising the amino acid sequence of SEQ ID NO:13 and a light chain variable region comprising the amino acid sequence of SEQ ID NO:9;
(b) a heavy chain variable region comprising the amino acid sequence of SEQ ID NO:14 and a light chain variable region comprising the amino acid sequence of SEQ ID NO:10;
(c) a heavy chain variable region comprising the amino acid sequence of SEQ ID NO:15 and a light chain variable region comprising the amino acid sequence of SEQ ID NO:11;
(d) a heavy chain variable region comprising the amino acid sequence of SEQ ID NO:16 and a light chain variable region comprising the amino acid sequence of SEQ ID NO:12;

In some embodiments, the humanized antibody or antigen binding portion thereof comprises a heavy chain variable region comprising an amino acid sequence selected from the group consisting of SEQ ID NOs:22-25 and a light chain variable region comprising an amino acid sequence selected from the group consisting of SEQ ID NOs:18-21. Preferred heavy and light chain combinations include but not limited to:
(a) a heavy chain variable region comprising the amino acid sequence of SEQ ID NO:22 and a light chain variable region comprising the amino acid sequence of SEQ ID NO:18;
(b) a heavy chain variable region comprising the amino acid sequence of SEQ ID NO:23 and a light chain variable region comprising the amino acid sequence of SEQ ID NO:19;
(c) a heavy chain variable region comprising the amino acid sequence of SEQ ID NO:24 and a light chain variable region comprising the amino acid sequence of SEQ ID NO:20;
(d) a heavy chain variable region comprising the amino acid sequence of SEQ ID NO:25 and a light chain variable region comprising the amino acid sequence of SEQ ID NO:21;

In some embodiments, the humanized anti-CD137 agonist antibody or antigen binding portion thereof comprises a light chain comprising an amino acid sequence selected from the group consisting of SEQ ID NOs:33, 37-39 and a heavy chain comprising an amino acid sequence selected from the group consisting of SEQ ID NOs:32, 34, and 40-42.

In some embodiments, the humanized anti-CD137 agonist antibody or antigen binding portion thereof comprises a light chain comprising an amino acid sequence selected from the group consisting of SEQ ID NOs:43-46 and a heavy chain comprising an amino acid sequence selected from the group consisting of SEQ ID NOs:47-50.

In some embodiments, the invention provides an anti-CD137 antibody, or an antigen-binding fragment thereof, comprising a heavy chain comprising a CDR3 region as set forth in any one of SEQ ID NOs:8 and 31, and comprising a heavy chain variable region comprising an amino acid sequence that has at least 95%, at least 96%, at least 97%, at least 98%, or at least 99% identical to a sequence as set forth in any one of SEQ ID NOs:2, 13-17, 22-25, and 36.

In some embodiments, the invention provides an anti-CD137 antibody, or an antigen-binding fragment thereof, comprising a light chain comprising a CDR3 region as set forth in any one of SEQ ID NOs:5 and 28, and having a light chain variable region comprising an amino acid sequence that has at least 95%, at least 96%, at least 97%, at least 98%, or at least 99% identical to a sequence as set forth in any one of SEQ ID NOs:1, 9-12, 18-21, and 35.

Thus, in certain embodiments, the CDR3 region is held constant, while variability may be introduced into the remaining CDRs and/or framework regions of the heavy and/or light chains, while the antibody, or antigen binding fragment thereof, retains the ability to bind to CD137 and retains the functional characteristics, e.g., binding affinity, of the parent.

In some embodiments, the substitutions made within a heavy or light chain that is at least 95% identical (or at least 96% identical, or at least 97% identical, or at least 98% identical, or at least 99% identical) are conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24:307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

The present disclosure also provides isolated polynucleotides (or nucleic acid molecules) encoding the various amino acid sequences (e.g., those of the antibodies or antigen-binding portion(s) thereof) disclosed herein, vectors containing the polynucleotides, cells (or host cells) containing the vectors, cells containing or expressing the various amino acid sequences disclosed herein, methods of making the antibodies (and/or fragments thereof), pharmaceutical compositions containing the antibodies disclosed herein, etc.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### Working Examples

The following examples are not intended to limit the scope of the claims to the invention, but is rather intended to be exemplary of certain embodiments. Any variations in the exemplified methods which occur to the skilled artisan are intended to fall within the scope of the present invention.

### Vector Construction

Vector pcDNA3.4TOPO (Invitrogen) was ligated to a short polylinker containing EcoRI, XhoI, and NotI. The resulting plasmid was digested with EcoRI and NotI restriction enzymes and purified by gel electrophoresis. For heavy chain cloning, the prepared vector was assembled using Gibson assembly the prepared vector, a gblock encoding and VH region (IDT), and human IgG2 gblock encoding an XhoI site at the junction of the J-chain and CH1 region. The plasmid was prepared and digested with EcoRI and XhoI to accommodate all the humanized variable heavy (VH) regions with an IgG2 isotype. All assembly was done with the Gibson method (NEB). Variable light regions were constructed with a similar method using gblocks to assemble Vkappa regions with a gblock fragment which encoded the constant kappa (Ck).

### Protein Expression

Plasmids were prepped and transfected into Expi293 or ExpiCHO cells using the transient expression system (ThermoFisher). Briefly, plasmids were transfected into 3e6 cells/ml cells at 1 μg plasmid DNA total/ml culture. The heavy chain and light chain plasmids were mixed at a 1:1ratio. Cultures were incubated at 37° C., shaking. After 16 hours, Transfection Enhancer 1 and 2 were added to the cultures and incubation was continued for six days. Supernatant was filtered, and protein titers were determined by an IgG quantitation protocol using the Octet Red96 (Pall). (Table 1) IgG was purified by Mab Select Sure Protein-A column purification on an ACTA PURE system and dialyzed overnight in PBS.

TABLE 1
Octet Data. Mono-valent Binding Kinetics of Anti-4-1BB Antibodies

| | | | | | T_{1/2} Life |
|---|---|---|---|---|---|
| | Response | KD (M) | kon(1/Ms) | kdis(1/s) | (min) |
| C1 | 0.1222 | 4.15E−08 | 2.79E+05 | 1.16E−02 | 1.0 |
| C2 | 0.1436 | 3.51E−08 | 1.62E+05 | 5.69E−03 | 2.0 |
| cAb 137QL.F1.1.G2 | 0.1384 | 4.19E−08 | 1.38E+05 | 5.78E−03 | 2.0 |
| cAB 137QL.G28.21.G2 | 0.2045 | 1.40E−08 | 3.73E+05 | 5.23E−03 | 2.2 |
| huG28.21.G2.1.4 | 0.1811 | 1.53E−08 | 4.26E+05 | 6.52E−03 | 1.8 |
| huG28.21.G2.4.4 | 0.1747 | 1.04E−08 | 4.49E+05 | 4.67E−03 | 2.5 |
| huF1.1.G2.2.4 | 0.1197 | 4.06E−08 | 1.38E+05 | 5.59E−03 | 2.1 |
| huF1.1.G2.4.4 | 0.1369 | 4.04E−08 | 1.53E+05 | 6.18E−03 | 1.9 |

### Flow Cytometry

Human/Cyno/Mouse 4-1BB transfected HEK293 cells or PHA-P stimulated PBMCs were incubated with serial diluted anti-4-1BB antibodies in FACS buffer (DPBS+ 2% FBS+ 0.05% sodium azide) followed by AF647-labeled F(ab′)2 goat anti-human IgG and 7-AAD. In addition, PBMCs were incubated with FITC Mouse anti-human CD3. Stained 4-1BB expressing HEK293 samples were analyzed using FlowJo by gating on FSC/SSC, followed by live/dead cell gating, and human/Cynomolgous/Mouse 4-1BB+ cells (MFI Geo Mean denoted). Stained Lymphocyte samples were analyzed using FlowJo by gating on FSC/SSC, followed by live/dead cell gating, and CD3+ cells with 4-1BB positives. (Table 2 and Table 3)

TABLE 2
Flow cytometry and NK-kB luciferase detection. Anti-4-1BB mAbs
bind to cell surface antigen. Anti-4-1BB mAbs do not bind to mouse
4-1BB or unstimulated human T Cells (data not shown). No NF-kB
stimulation was observed in the absence of Fc cross-linking.

| Cell Binding EC50 (□g/ml) | | | | |
|---|---|---|---|---|
| | HEK293 cells | HEK293 cells | | NF-□B |
| | over-expressing | over-expressing | Activated | Stimulation |
| | human 4-1BB | cyno 4-1BB | T cells | (EC50 □g/ml) |
| F1.1 | 0.341 | 0.392 | 0.233 | 0.026 |
| G28.21 | 0.121 | 0.162 | 0.080 | 0.018 |
| C1 | 0.105 | 0.168 | 0.048 | 0.032 |

TABLE 3
Flow cytometry. Anti-4-1BB Antibodies
bind to cell surface antigen.

| | HEK293 cells | HEK293 cells | |
|---|---|---|---|
| | over-expressing | over-expressing | Activated |
| | human 4-1BB | cyno 4-1BB | T cells |
| Chimeric F1.1 | 0.202 | 0.309 | |
| Chimeric | 0.056 | 0.099 | |
| G28.21 | | | |
| huF1.1.2.4 | 0.404 | 0.602 | 0.234 |
| huF1.1.4.4 | 0.379 | 0.545 | 0.146 |
| huG28.21.1.4 | 0.083 | 0.154 | 0.010 |
| huG28.21.4.4 | 0.086 | 0.135 | 0.010 |
| C1 | 0.053 | 0.091 | ~to G28.21 |
| C2 | 0.051 | No Binding | 0.026 |

### NF-κB Reporter Assay Using Human 4-1BB 293 Transfected Cells with Fc-Cross-Linking From CHOK1hFcgRIIA/CHOK1 Cells

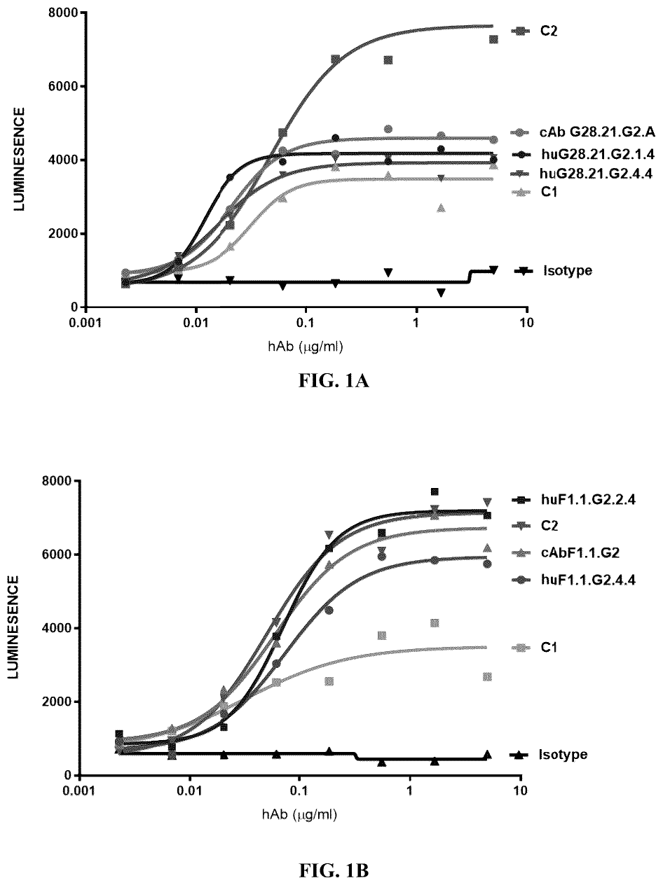

NF-κB Hu 4-1BB 293 transfected cells were made by transfecting HEK-Dual™ TNF-α cells (Invivogen) with 4-1BB and were used to measure TNF-α induced NF-kB activation. hFcgRIIA/CHOK1 transfected cells were used to provide cross-linking to anti-4-1BB IgGs. Cells were harvested with Accutase, washed, and resuspended with DMEM without phenol red +10% heat-inactivated Fetal Bovine Serum at 1×10⁶ cells/ml. 5×10⁴ NF-κB 4-1BB 293 transfected cells were co-cultured in Costar 3799 U-bottom 96-well plates with an equal number of hFcgRIIA/CHOK1 transfected cells or CHOK1 parental cells. Test antibodies, positive control antibodies (C1 & C2), and negative control (Isotype) were serially diluted in media and added to the cells. Following incubation at 37° C. for 18-22 hours, 20 μl of cell suspension was removed from the wells and mixed with 50 μl of luminescence assay reagent (Quanti-Luc, Invivo-Gen #rep-qlc) in an opaque 96 flat-bottom plate. Luminescence (RLUs) was measured on a Flexstation3 plate reader with integration of 100 ms. (FIGS. 1A and 1B) (Table 4).

TABLE 4
NF-KB reporter activation is induced by anti-4-1BB (anti-CD137) agonist antibody.

| | huF1.1.G2.2.4 | huF1.1.G2.4.4 | cAb F1.1.G2 | C2 | C1 |
|---|---|---|---|---|---|
| Bottom | 838 | 666 | 894 | 521 | 669 |
| Top | 7194 | 5942 | 6730 | 7140 | 3504 |
| HillSlope | 1.756 | 1.300 | 1.285 | 1.312 | 0.935 |
| EC50 | 0.071 | 0.074 | 0.060 | 0.050 | 0.033 |
| | huG28.21.G2.1.4 | huG28.21.G2.4.4 | cAb G28.21.G2 | C2 | C1 |
| Bottom | 625 | 592 | 879 | 492 | 933 |
| Top | 4173 | 3924 | 4592 | 7655 | 3482 |
| HillSlope | 2.715 | 1.546 | 1.845 | 1.290 | 2.336 |
| EC50 | 0.012 | 0.016 | 0.021 | 0.047 | 0.031 |

### Anti-4-1BB Mediated T Cell Proliferation & Interferon Gamma Release Cross-Linked with CHOK1mFcgRIIB Transfected Cells

T cells were prepared from Peripheral blood mononuclear cells (PBMCs) with the Pan T cell Isolation kit (Miltenyi Biotec product #130-096-535), resuspended in cold PBS, 0.5% bovine serum albumin (BSA) and 2 mM EDTA, pH 7.2, and labeled with Cell Trace Violet (ThermoFisher, cat #C34557) per kit instructions. Finally, labeled pan T cells were resuspended in Advanced

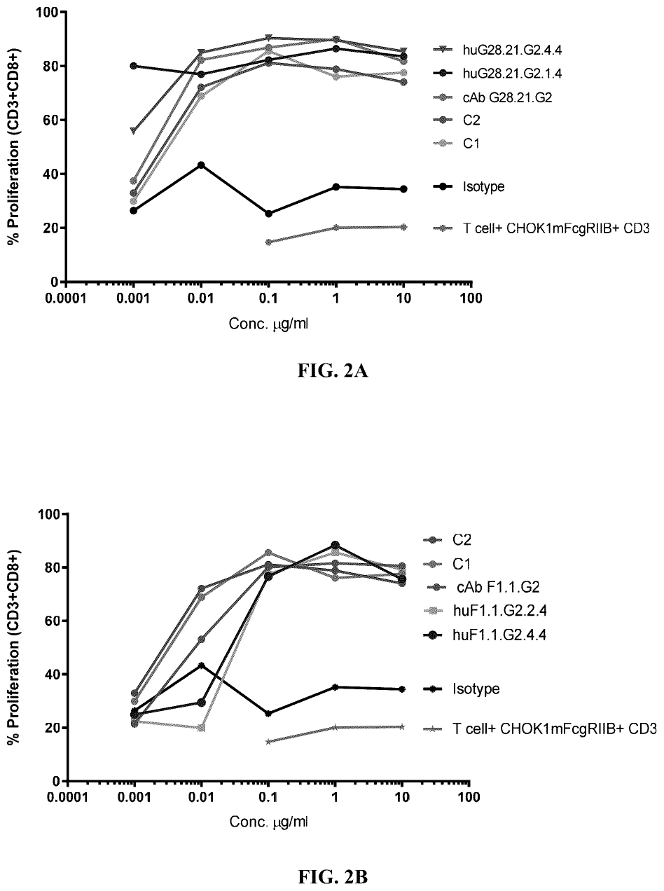

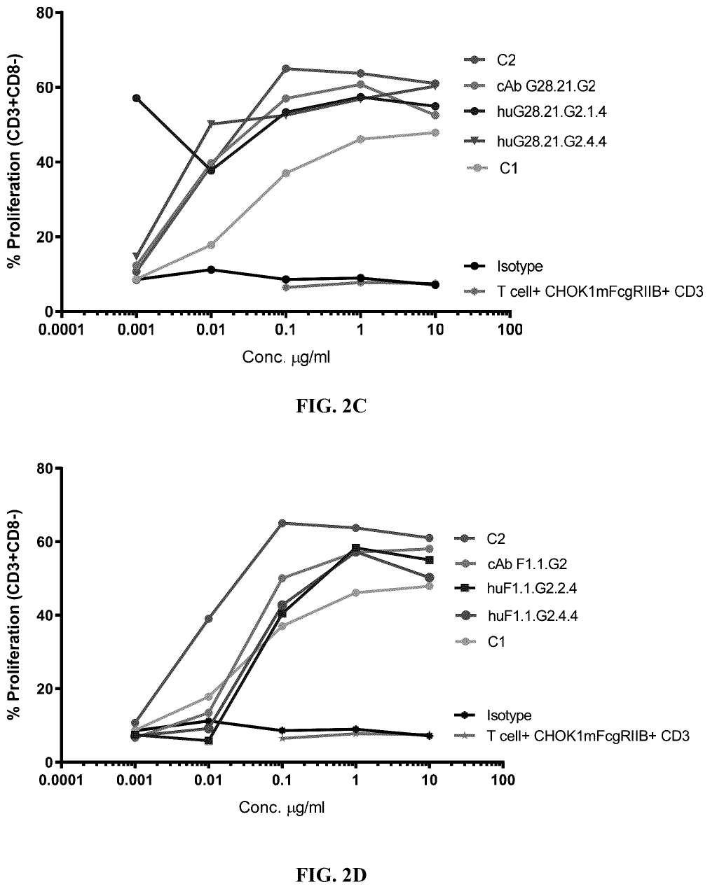

RPMI-1640, 10% heat-inactivated FBS, 2-Mercaptoethanol (1:1000) at 2×10⁶ cells/ml. CHOK1.mFcgRIIB-mCherry cells were cultured in F12 Ham, 10% FBS, 5 μg/ml Puromycin, harvested by Accutase, and resuspended in Advanced RPMI-1640, 10% heat-inactivated FBS, 2-Mercaptoethanol (1:1000) at 2×10⁵ cells/ml. CHOK1 parental cells were prepared similarly without Puromycin in the media. To a 96-wells U-bottom culture plate, 100,000 labeled pan T cells/well were mixed with CHOK1mFcgRIIB-mCherry or CHOK1 at 10,000 cells/well, a 10:1 ratio. 10 ng/ml NA/LE Mouse anti-Human CD3 (clone UCHT-1) was added to all wells, and then serial diluted 4-1BB test antibodies, positive control antibody (C1 or C2), and negative control antibody (Isotype) at a starting concentration of 1 μg/ml were added. Control wells included CHOK1mFcgRIIB-mCherry, Pan T cells+CHOK1mFcgRIIB, Pan T cells+CHOK1mFcgRIIB+anti-human CD3, Pan T cells+anti-human CD3, and Pan T cells alone. Plates were incubated for 5 days in a 37° C. CO₂ incubator. Cells were further stained for flow cytometry with FITC anti-huCD3, PE anti-huCD8, and 7-AAD. Samples were analyzed using FlowJo by gating on FSC/SSC, followed by live/dead cell gating, then proliferation (CellTrace Violet) of CD3+/CD8+ T cells and CD3+/CD8− T cells. FIGS. 2A and 2B show anti-4-1BB (anti-CD137) agonist antibody stimulates primary CD3+ CD8+ T cell proliferation. FIG. 2C and 2D show anti-4-1BB (anti-CD137) agonist antibody stimulates primary CD3+ CD8− T cell proliferation.

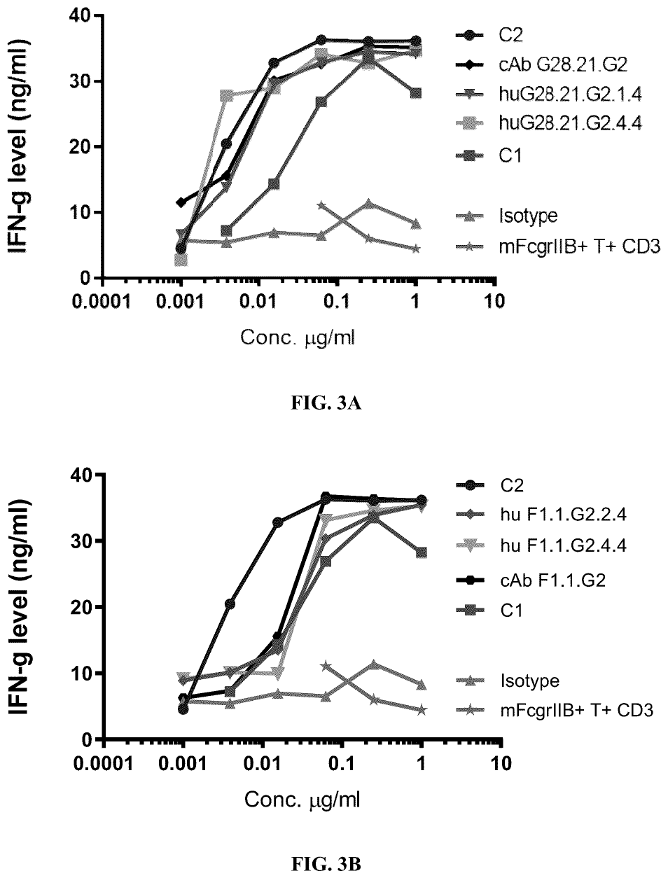

The cytokine (IFN-γ) release assay was similarly prepared without pan T cell labeling. Plates were incubated 48 hours in the 37° C. CO₂ incubator and culture supernatants were separated and collected onto two U-bottom 96-wells plates (70-80 μl/plate). For Elisa, culture supernatants were diluted 1:2 with Reagent diluent (R&D Systems, cat #DY995). Refer to R&D Systems Data Sheet and Assay Procedure (product #DY285B). FIGS. 3A and 3B show anti-4-1BB (anti-CD137) agonist antibody stimulates interferon-γ secretion.

### MC38 Growth Inhibition in Transgenic Human 4-1BB Knock-in Mice

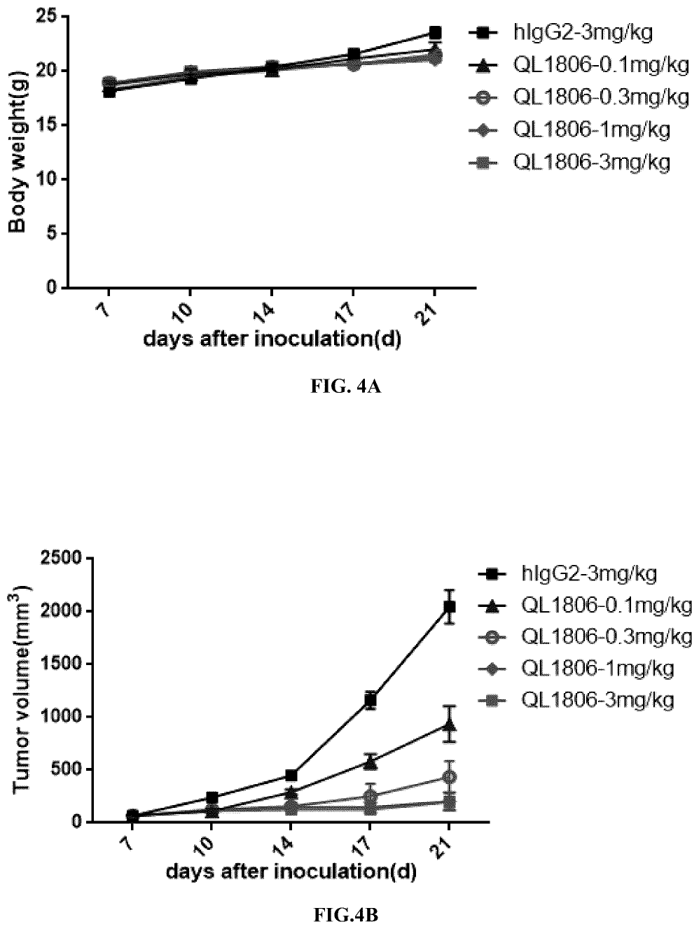

Mouse colon cancer MC38 cells were expanded in DMEM medium containing 10% fetal bovine serum, 1% penicillin and 1% streptomycin and 2 mM glutamine in a 37° C., 5% CO₂ incubator. 5E5 MC38 cells were seeded s.c. into the right armpit and tumors grew to 50-100 mm³ prior to administration of the test article. Per group, eight h4-1BB knock-in female mice were administered anti-4-1BB antibody at 0.1, 0.3, 1, or 3 mg/kg or hIgG2 at 3 mg/kg, i.p., twice per week for a total of 5 doses. FIG. 4A show that body weight of all test transgenic hu4-1BB knock-in mice gradually increased following administration of anti-4-1BB antibody. In addition, no drug-related toxicity was observed. QL1806 represents the anti-4-1BB antibody. FIG. 4B shows anti-4-1BB inhibits the growth of mouse colon cancer cells MC38. At day 21, significant inhibition (p<0.01) was observed and the 1 mg/kg and 3 mg/kg dose groups contained two tumor-free mice each (Table 5).

TABLE 5
Day 21 Tumor volume, inhibition rate,
and tumor regression (n = 8)

| | Tumor volume | TGI | T/C | Tumor |
|---|---|---|---|---|
| Group | (mm³) | (%) | (%) | free |
| hIgG2 (3 mg/kg) | 2048 ± 158.8 | / | / | 0/8 |
| QL1806 | 935.8 ± 167.9 ** | 56.2 | 45.3 | 0/8 |
| (0.1 mg/kg) | | | | |
| QL1806 | 433.4 ± 152.4 ** | 81.5 | 21.6 | 1/8 |
| (0.3 mg/kg | | | | |
| QL1806 | 204.1 ± 84.5 ** | 93.1 | 9.91 | 2/8 |
| (1 mg/kg) | | | | |
| QL1806 | 197.1 ± 50.9 ** | 93.5 | 9.62 | 2/8 |
| (3 mg/kg) | | | | |

Note:
Compared with hIgG2 (3 mg/kg group),
* p < 0.05;
** p < 0.01.

The clone names (antibodies) shown in the figures, tables, and examples described herein comprise the heavy chain and light chain pairings shown in Table 6 below:

TABLE 6
Sequences for Clone Names

| Name | Heavy Chain SEQ ID NO | Light Chain SEQ ID NO |
|---|---|---|
| G28.21 | 2 | 1 |
| F1.1 | 36 | 35 |
| huG28.21.G2.1.4 | 32 | 33 |
| huG28.21.G2.4.4 | 34 | 33 |
| huF1.1.G2.2.4 | 48 | 46 |
| huF1.1.G2.4.4 | 50 | 46 |
| QL1806 | 34 | 33 |

We claim:

1. An isolated anti-CD137 agonist antibody, or an antigen-binding portion thereof comprising a heavy chain variable region CDR3 comprising SEQ ID NO:8.

2. An isolated anti-CD137 agonist antibody, or an antigen-binding portion thereof comprising a heavy chain variable region CDR3 comprising SEQ ID NO:31.

3. The isolated antibody, or an antigen-binding portion thereof, of claim 1, which further comprises a heavy chain variable region CDR1 comprising SEQ ID NO:6 and a heavy chain variable region CDR2 comprising SEQ ID NO:7 respectively.

4. The isolated antibody, or an antigen-binding portion thereof, of claim 2, which further comprises a heavy chain variable region CDR1 comprising SEQ ID NO:29 and a heavy chain variable region CDR2 comprising SEQ ID NO:30 respectively.

5. The isolated antibody, or an antigen-binding portion thereof, of claim 3, which further comprises
(a) a light chain variable region CDR1 comprising SEQ ID NO:3;
(b) a light chain variable region CDR2 comprising SEQ ID NO:4; and
(c) a light chain variable region CDR3 comprising SEQ ID NO:5;
wherein said antibody or portion specifically binds to human CD137.

6. The isolated antibody, or an antigen-binding portion thereof, of claim 4, which further comprises
(a) a light chain variable region CDR1 comprising SEQ ID NO:26;
(b) a light chain variable region CDR2 comprising SEQ ID NO:27; and
(c) a light chain variable region CDR3 comprising SEQ ID NO:28;
wherein said antibody or portion specifically binds to human CD137.

7. The isolated antibody, or antigen-binding portion thereof, of claim 1, which comprises a light chain variable region amino acid sequence having at least 95% identity to SEQ ID NO:1 and a heavy chain variable region amino acid sequence having at least 95% identity to SEQ ID NO:2.

8. The isolated antibody, or an antigen-binding portion thereof, of claim 1, which comprises a heavy chain variable region comprising an amino acid sequence that has at least 95%, at least 96%, at least 97%, at least 98%, or at least 99% identical to a sequence as set forth in SEQ ID NO:2.

9. The isolated antibody, or antigen-binding portion thereof, of claim 2, which comprises a light chain variable region amino acid sequence having at least 95% identity to SEQ ID NO:35 and a heavy chain variable region amino acid sequence having at least 95% identity to SEQ ID NO:36.

10. The isolated antibody, or an antigen-binding portion thereof, of claim 2, which comprises a heavy chain variable region comprising an amino acid sequence that has at least 95%, at least 96%, at least 97%, at least 98%, or at least 99% identical to a sequence as set forth in SEQ ID NO:36.

11. The isolated antibody, or antigen-binding portion thereof, of any one of claims 1-10, which is a Fab fragment, an F(ab′)₂ fragment, an Fv fragment, a single chain antibody, or a bispecific antibody.

12. The isolated antibody of any one of claims 1-10, which is a chimeric antibody or humanized antibody.

13. The isolated antibody of any one of claims 1-10, which is an immunoglobulin G (IgG), an IgM, an IgE, an IgA or an IgD molecule.

14. The isolated antibody of claim 13, which is an IgG1, IgG2, IgG3, or IgG4.

15. The isolated antibody, or antigen-binding portion thereof, of claim 1, which comprises: a heavy chain variable region comprising an amino acid sequence selected from the group consisting of SEQ ID NOs: 13-17 and a light chain variable region comprising an amino acid sequence selected from the group consisting of SEQ ID NOs:9-12.

16. The isolated antibody, or antigen-binding portion thereof, of claim 2, which comprises: heavy chain variable region comprising an amino acid sequence selected from the group consisting of SEQ ID NOs:22-25 and a light chain variable region comprising an amino acid sequence selected from the group consisting of SEQ ID NOs:18-21.

17. The isolated antibody of any one of claims 15 and 16, which is a chimeric antibody or humanized antibody.

18. The isolated antibody of any one of claims 15 and 16, which is an immunoglobulin G (IgG), an IgM, an IgE, an IgA or an IgD molecule.

19. The isolated antibody of claim 18, which is an IgG1, IgG2, IgG3, or IgG4.

20. The isolated antibody of any of claims 1-19, wherein the antibody is monoclonal.

21. An isolated monoclonal antibody or antigen binding portion thereof, comprising a light chain comprising an amino acid sequence selected from the group consisting of SEQ ID NOs:33, 37-39 and a heavy chain comprising an amino acid sequence selected from the group consisting of SEQ ID NOs:32, 34, 40-42.

22. An isolated monoclonal antibody or antigen binding portion thereof, comprising a light chain comprising an amino acid sequence selected from the group consisting of SEQ ID NOs:43-46 and a heavy chain comprising an amino acid sequence selected from the group consisting of SEQ ID NOs:47-50.

23. An immunoconjugate comprising the antibody, or antigen-binding portion thereof, of any one of claims 1-10, 15, 16, 21 and 22, linked to a therapeutic agent.

24. A pharmaceutical composition comprising the antibody, or antigen-binding portion thereof, according to any one of claims 1-10, 15, 16, 21 and 22 and a pharmaceutically acceptable carrier.

25. A method of stimulating immune responses in a subject, comprising the step of administering to the subject the pharmaceutical composition according to claim 24 in therapeutically effective amount to stimulate an immune response in said subject.

26. A method of treating infectious diseases in a subject, comprising the step of administering to the subject the pharmaceutical composition according to claim 24 in therapeutically effective amount to treat said infectious diseases.

27. A method of treating cancer in a subject, comprising the step of administering to the subject the pharmaceutical composition according to claim 24 in therapeutically effective amount to treat said cancer.
